(19) United States
(12) Patent Application Publication
(10) Pub. No.: US 2011/0276522 A1
(43) Pub. Date: Nov. 10, 2011

(54) Standardization and reconstitution of phytochemicals for medical dispensation

(76) Inventors: Gary S. Kehoe, Glendale, AZ (US); Gregory W. Gossett, Ogden, UT (US)

(21) Appl. No.: 13/065,776

(22) Filed: Mar. 30, 2011

Related U.S. Application Data
(60) Provisional application No. 61/341,424, filed on Mar. 31, 2010.

(51) Int. Cl. G06Q 90/00; A61K 36/00

(52) U.S. Cl. 705/500; 424/725

(57) ABSTRACT

A method to produce compositions with a standardized phytochemical content comprises growing marijuana in a controlled environment, harvesting live green leaves from the marijuana, processing the green leaves to produce a phytochemically standardized slurry, heating the slurry to produce a thin film, and cutting a film into pieces each sized to contain a selected dosage of the phytochemical.

This application claims priority based on U. S. Provisional Patent Application Ser. No. 61/341,424 filed Mar. 31, 2010.

This invention relates to the use of boactive phytochemicals in the medical treatment of various ailments.

More particularly, the invention relates to the beneficial use of bioactive phytochemicals found in marijuana.

The use of marijuana is legally circumscribed in many states. It has been, however, well known that there are bioactive phytochemicals in marijuana that are useful in the treatment of medical ailments. Those of skill in the art have long sought improved methods of utilizing such phytochemicals while adhering to limitations prescribed by the law. Accordingly, it would be highly desirable to provide an improved process to facilitate the beneficial use of marijuana phytochemicals in harmony with various state and federal criminal statutes.

We have discovered a new process for producing and dispensing a beneficial bioactive phytochemical found in a marijuana plant. The process comprises the steps of selecting an area of restricted size in which to grow marijuana; growing marijuana in said the area; harvesting live green marijuana leaves from plants being grown in the selected area; determining and recording in a first tangible inventory file the quantity of marijuana leaves harvested; mechanically grinding, pulverizing, and homogenizing the harvested live marijuana leaves to produce a slurry with particulate sufficiently small to pass through openings no larger than those found in a twenty-five mesh screen, the slurry including natural hemp fibers and the beneficial bioactive phytochemical found in the marijuana plants and uniformly distributed throughout the slurry; determining the concentration in the slurry of the beneficial bioactive phytochemical; adding, if necessary, to the slurry an additional quantity of the beneficial bioactive phytochemical to achieve a desired concentration in the slurry of the phytochemical; dispensing the slurry on a drying surface; moving the drying surface and slurry through an oven to remove moisture from said slurry and produce a thin film including natural hemp fibers and the beneficial bioactive phytochemical; cutting the thin film into film pieces each having a selected dosage of the beneficial bioactive phytochemical; packaging the film pieces to produce thin film packages each including a selected number of the film pieces; recording in a second tangible inventory file the number of the thin film packages produced; selling the thin film packages; recording in a third tangible inventory file the number of thin film packages sold and paying tax on each of the thin film packages sold; and, auditing the first, second, and third inventory files.

The area in which the marijuana is grown can be selected as desired and can be an enclosed indoors area or can be an area out-of-doors. An indoor area of a selected restricted size is presently preferred since it facilitates control of the growing environment and maintaining records of plants grown and harvested. For example, if an indoors area is utilized, the number of shelves on which marijuana plants are grown can more readily be defined, as can the number of plants grown on each shelf. In time, data gathered can be used to evaluate whether the number of plants and the quantity of marijuana leaves harvested that are reported by a grower is reasonable. An indoors area likely would utilize growth lights to expedite the growth of marijuana plants. Air conditioning and heating may be utilized as desired. When an out-of-doors area is selected, it may be necessary to determine an estimated number of plants or leaves per acre, half-acre, quarter-acre, etc. and to use that to account for the number of plants and for the number of leaves harvested. In one embodiment of the invention, the entity growing marijuana in a selected area is licensed to do so by an appropriate city, county, state, and/or federal authority. The area where marijuana is grown preferably must have minimal security protocol, possibly including being locked when not occupied by an individual, including burglar alarm systems, including entry systems requiring fingerprint identification, including security cards, etc. CGMP (common good manufacturing practices) preferably are utilized during the growth of marijuana plants, during the harvesting and processing of the plants, during shipping, and during payment of any taxes due governmental authorities.

After the marijuana plants are grown in a particular area, an inventory of the number of plants is taken and is recorded in a first inventory file. In some instances, as noted above, the number of plants in an indoors or out-of-doors area can be estimated (possibly by calculating a number of plants or weight of marijuana leaves produced per square foot, for example), particularly when the areas utilized are large.

Live green leaves from the marijuana plants are harvested in their natural green live state after the plants have grown to a desired size, or have grown for a selected period of time. The quantity of leaves harvested is recorded in a second inventory file. The quantity of leaves can, by way of example and not limitation, consist of the number of leaves harvested or consist of the total weight of the plant leaves harvested. If desired, both the first and second inventory files are not utilized, and only one of said inventory files is used and maintained.

In one embodiment of the invention, marijuana plants are grown in a warehouse on racks equipped with spaced apart horizontally oriented shelves. The warehouse can be one or more stories high. The warehouse usually, but not necessarily, is enclosed and is air conditioned or heated, as appropriate. Wooden pallets are utilized. Each pallet typically is four feet by four feet and is of the type that is utilized by a fork lift to move about products that are stored on the pallet. A planter is constructed on top of each pallet, and is filled with soil. The bottom of the planter is provided with a drainage system. The drainage system can, for example, comprise a layer a gravel and drainage holes formed in the bottom of the planter or can comprise a sewer pipe or other conduit that is perforated. The sewer pipe is canted such that excess water will flow out from the bottom of the planter and through a side of the planter into a gutter or other water removal system used in connection with the planter. Each pallet-planter unit is moved about with a fork lift that engages the pallet. Multiple shelving racks, each with tiers of spaced apart horizontally oriented shelves, are constructed in the warehouse. A fork lift is used to place one or more pallet-planter units on each horizontally oriented level or tier of shelving racks. Lighting is provided on the underside of each shelving level, or at other desired locations, such that the plants in each pallet-planter unit on a level of the shelving racks continuously receive light. The lighting preferably comprises LED light panels. A spray or other watering system is providing for each level of shelving to automatically water each planter at a selected time(s) of day. A fork lift removes pallet-planter units from shelving to harvest marijuana leaves, places pallet-planter units on shelving after marijuana plants or seeds are planted in the units, and moves pallet-planter units to and from the shelving as otherwise desired. Lighting, fertilization, and pruning can each affect the growth rate and leaf production of a marijuana plant, as well as the period of time for which a plant has been growing. It is likely that an expected baseline yield of leaves per pallet-planter unit in terms of pounds can be estimated for each pallet-planter unit and can be confirmed as marijuana harvests are monitored over a period of time. This expected yield can be compared to reported yields to provide a rough guideline as to whether reported yields are reasonable. If desired, the baseline expected yield per pallet can comprise the second inventory. Clipping the ends off a marijuana branch at appropriate times during the growth of marijuana plants can produce multiple additional shoots or branches on the remaining length of the clipped branch and increase the yield of marijuana leaves.

The natural green living leaves (i.e., not dried) are harvested off the stalks of the marijuana plants. The live green leaves are mechanically cut, ground, and/or pulverized and are then homogenized to produce a slurry with particulate sufficiently small to pass through a screen having openings no larger than those in a 25 mesh screen, preferably no larger than those in a 30 mesh screen. A screen with openings larger than those in a 25 mesh screen is not preferred. A screen with openings smaller than those a 50 mesh screen also is not preferred. Accordingly, screens in the range of 25 mesh to 50 mesh are utilized. The slurry that is produced includes natural hemp fibers and various beneficial bioactive phytochemicals that are found in the marijuana plant. Water or another desired liquid can be added while the harvested marijuana plants are ground, cut, and/or pulverized, and are homogenized to produce the slurry. Apart from water or another liquid which may be added to the slurry, the slurry preferably only consists of the ground, pulverized, and homogenized marijuana plants. Further still, the marijuana plants preferably are organically grown. The above-noted cutting, grinding, pulverizing, homogenizing process avoids the time consuming process of drying marijuana plants. The amount of water added to the slurry can, if necessary, be varied as desired. But in one embodiment of the invention that produces a thin film, sufficient water is added to facilitate homogenizing a marijuana plant or leaves, but that at the same time minimizes the amount of water that must be evaporated to produce a thin film. The slurry is characterized as a suspension because hemp fibers are suspended in he slurry. A gel can be produced from the slurry by adding pectin or other desired thickeners.

In one embodiment of the invention, the leaves of natural green live marijuana plants are processed to produce a fine fibrous homogeneous slurry. This is accomplished by rough grinding and pulverizing the leaves for about ten minutes or until they can pass through a ten mesh screen, and by then homogenizing the leaves using a rotating blade at a minimum of two hundred RPM for no less than twenty minutes to homogenize the leaf slurry until they will pass through a thirty mesh screen. During this process water is, if necessary, added as desired to facilitate the production of a fine fibrous homogeneous liquid.

In an alternate embodiment of the invention, the marijuana leaves are dried for a selected period of time prior to being ground, pulverized and homogenized.

The concentration in the slurry of the desired bioactive phytochemical(s), for example THC, is determined. This concentration typically is determined after all flavorings, coloring, etc. has been added and admixed to the slurry. If it is desired to increase the concentration of the desired bioactive phytochemical to a selected level necessary to produce an end product with a selected standardized concentration of the phytochemical, a quantity of the bioactive sufficient to produce the desired concentration of bioactive phytochemical in the slurry is added to the slurry. In the event a quantity of phytochemical such as THC is added to the slurry to increase the concentration of THC in the slurry, various methods are known for extracting phytochemicals such as THC from marijuana to supply the THC that must be added to the slurry. These methods are not detailed herein. In order to facilitate the incorporation in the slurry of an additional quantity of a phytochemical, a surfactant, emollient, flavor oil, or other desired component can be utilized.

The slurry is dispensed on a drying surface such as, for example, the flat surface of a cookie sheet. The cookie sheet is heated to evaporate water from the slurry to produce a layer of thin film. In one embodiment of the invention, the sheet is heated by placing it in the ambient air. In another embodiment of the invention, the sheet is heated by moving the sheet through a drying oven. The layer of thin film that is produced includes natural hemp fibers and the desired bioactive phytochemical. Since the slurry was mixed homogeneously, the concentration of the phytochemical in one area of the thin film is substantially equivalent to the concentration of the phytochemical in another area of the thin film.

During cutting, pulverizing, or grinding marijuana leaves to produce a slurry, a nitrogen blanket can be utilized to eliminate any potential oxidation during such production. A vacuum step can be incorporate to remove oxygen or air from a slurry to eliminate any trapped oxygen in the finished thin film strip. Or, in contrast, the slurry can be aerated prior to application on a substrate for drying so that the density of the resulting thin film is reduced and the thin film dissolves more quickly in a user's mouth.

The thin film has a substantially uniform thickness throughout. The thin film is cut into film pieces of equivalent size. Each film piece has a standardized dosage, or concentration, of at least one reconstituted beneficial phytochemical in the film piece, that is substantially equivalent to the dosage of the reconstituted beneficial phytochemical in film pieces of equivalent size. In order to achieve different standardized dosages of a desired reconstituted beneficial marijuana phytochemical, film pieces of different size can, if desired, be cut from the thin film. Another method of achieving a different standardized dosage in a piece of thin film is, as described above, to alter in the slurry the concentration of a desired phytochemical.

The number of film pieces produced is recorded in a third inventory file. Each film piece contains a selected standardize dosage on concentration of at least one beneficial phytochemical found in marijuana. Presently, each phytochemically standardized film piece contains 0.25% to 50% by weight of at least one beneficial marijuana phytochemical, preferably 0.5 to 5% by weight of a beneficial marijuana phytochemical, and most preferably 0.75% to 2% by weight of a beneficial marijuana phytochemical. In another embodiment of the invention, each film piece contains 0.05% to 50% by weight, preferably 0.25% to 10% by weight, most preferably 0.75% to 7.5% by weight of at least one beneficial marijuana phytochemical. The dosage of a phytochemical, for example THC, in a film piece is in the range of 0.1 to 50 mg, preferably 2.0 mg to 12.5 mg, and most preferably 5.0 mg to 10 mg.

The film pieces are packaged in small containers called cassettes, are packaged in foil packets, or are otherwise packaged. The number of film pieces in each package can vary as desired. The number of packages of film pieces produced is recorded in a fourth inventory file, along with the number of film pieces in each cassette. If desired, only the third or fourth inventory file noted above is utilized, not both.

Each cassette has a lot number, expiration date, and tax stamp. A lot number is tracked back to a group comprising one or more pallet-planter units.

When the thin film packages are sold, the number of packages sold is recorded in a fifth inventory file, and city, county, state, and/or federal taxes—as appropriate—are paid on each of the thin film packages sold. The amount of the taxes is also recorded in the fifth inventory file.

In an alternate embodiment of the invention, tax is paid to a governmental authority on each thin film cassette or another alternate container of a standardized marijuana composition before the package leaves the factory or other production facility. One alternate container comprises a bottle containing the slurry produced above. A second alternate container comprises a foil package containing thin film which has been shredded to produce a product having a consistency like tobacco used in a cigarette or pipe.

The tax paid on each container of reconstituted marijuana—regardless of whether the container contains thin film, contains slurry, or contains some other form of phytochemically standardized reconstituted marijuana—can be calculated as desired.

The tax can be calculated based on the wholesale or retail sale price of the package.

Or, the tax can be calculated based on the number of doses the package contains based on a doctor's prescription to the consumer purchasing the package. If the doctor's prescription sets forth a dose as one mg of THC, if the package contains twenty thin films each containing one mg of THC, if the tax is five cents per mg dose of THC, then the tax on the package would be $1.00, i.e., $0.05 times twenty doses in the package.

Or, the tax can be based on the quantity of a particular phytochemical contained in the package. For example, the tax could be five cents per milligram of THC contained in the package.

Or, the tax can be based on the assumption that each square foot of growing space will produce so many pounds of marijuana leaves during each growing cycle. For example, if a growing cycle is twenty-one days, if the assumption is that each square foot will produce three pounds of marijuana leaves, if the growing space is 100 square feet, and if the tax is $2.00 per pound of marijuana, then the tax payable each twenty-one days is (100 sq. ft.)×(3 lbs/sq. ft)×(2 dollars/lb)=$600.00.

And so on.

The thin film pieces can be smoked or ingested, with ingestion being preferred. Ingestion of thin film pieces is preferably accomplished by dissolving a film piece in an individual's mouth to facilitate transmucosal delivery. Transmucosal delivery is preferred because such membranes are very thin and permeable and permit the gastrointestinal tract and first pass liver metabolism to be bypassed. The thin film can be lit with a flame and smoked by inhaling the smoke produced.

The first, second, third, fourth, and/or fifth inventory files are periodically audited to minimize the risk that beneficial phytochemicals or marijuana plants are being sold or distributed through improper channels. The first, second, third, fourth, and fifth inventory files can comprise files maintained on a computer, on punch cards, on magnetic tape, in bookkeeping books or journals, or in any other desired tangible record keeping source.

In another embodiment of the invention, a doctor's prescription must be obtained by a consumer before a package of thin film pieces containing a beneficial marijuana phytochemical is sold to the consumer. The consumer turns in the prescription at a drug store or other dispensing facility to obtain a package of thin film pieces, or to obtain a spray dispenser with a beneficial marijuana phytochemical, or to obtain a gel containing the phytochemical, etc.

In a further embodiment of the invention, THC is extracted from the slurry that is produced by mechanically grinding and homogenizing green marijuana plant. The THC is placed in a phytochemically standardized solution in water or another desired liquid and is placed in a pump spray bottle such that one or two displacements (or “pumps”) of the pump delivers a quantity of spray having a desired dose, say five mg, of a beneficial marijuana phytochemical. The spray can be administered to an individual orally or into nasal passages.

In still another embodiment of the invention, one or more beneficial phytochemicals is extracted from the slurry that is produced by mechanically grinding and homogenizing green marijuana plants. The extracted phytochemical is sold separately. The extracted phytochemical typically is concentrated prior to being sold. The extracted phytochemical can be incorporated and sold in a gel. Gels can be incorporated in the production of sprays, time release lozenges, reservoir packets, and other oral/buccal/transmucosal delivery systems. One example of an oral/buccal transmucosal system that can be used to deliver a marijuana phytochemical is the mini “tea bag” type device used by Skoal for its Skoal Bandits and Marlboro.

The total number of cannabinoids is marijuana is currently placed at about eighty. Each of these cannadinoids may provide advantageous medical treatment alternatives. In particular, other active phytochemical compounds in marijuana can be used to create reconstituted standardized thin film pieces or other standardized delivery systems along the lines that are described above include ⁹-Tetrahydrocannabinol (⁹-THC), ⁸-Tetrahydrocannadinol (⁸-THC), Cannabichromene (CBC), Cannabicyclol (CBL), Cannabidiol (CBD), Cannabielsion (CBE), Cannabigerol (CBG), Cannabinidiol (CBND), Cannabinol (CBN), and Cannabitriol (CBT). Each of these other active phytochemical compounds found in marijuana can be present in a phytochemically standardized film piece such that the film piece contains 0.25% to 50% by weight of the phytochemical compound, preferably 0.5 to 5% by weight of the phytochemical compound, and most preferably 0.75% to 2% by weight of the phytochemical compound. In another embodiment of the invention, each film piece contains 0.05% to 50% by weight, preferably 0.25% to 10% by weight, most preferably 0.75% to 7.5% by weight of the phytochemical compound. The dosage of one of the other phytochemical compounds in a film piece (or in the spray delivered by one pump of a spray bottle, or in another desired delivery system) of reconstituted marijuana is in the range of 0.1 to 50 mg, preferably 2.0 mg to 12.5 mg, and most preferably 5.0 mg to 10 mg.

A phytochemically standardized thin film produced in the manner set forth above can include edible film formers which change the rate of dissolution of the film in the mouth, which produce desired oral feel, or which affect the rate at which the film burns. In one embodiment of the invention, the thin film quickly dissolves in the oral cavity within five to ten seconds to deliver rapidly the entire dosage of a phytochemical. In another embodiment of the invention, the thin film gradually dissolves in the oral cavity over a selected period of time.

Any desired bioactive or flavoring can be incorporated in a thin film or liquid form of reconstituted marijuana which contains a desired dose of a beneficial marijuana phytochemical. The viscosity of a marijuana derived film or liquid can vary as desired.

A thin film produced in accordance with the invention can include one or more bulking agents, softeners, intense artificial sweeteners, sugar alcohols, natural sweeteners, flavorings, cooling agents, surfactants, coloring agents, oils, and drying agents.

A thin film produced in accordance with the invention can incorporate one or more film formers such as, by way of example and not limitation, pullulan, guar gum, pectin, xanthan gum, alginates, gelatin, starches (including corn, potato, rice or tapioca), modified starches, maltodextrins, wheat gluten, carboxymethylcellulose, carrageenan, konjac, or locust bean gum.

The naturally derived gel from the marijuana plant is water miscible and can be modified if mixed with other bulking agents, film formers, humectants, softeners, flavorings, and sweeteners. The soluble gel matrix is then applied to a film substrate by way of an extrusion die or by using a knife over roller method. The coated film is then dried, slit into strips, and cut into desired lengths.

When a phytochemically standardized thin film is formed in accordance with the processes described above, the weight of each individual thin film piece that is produced preferably, but not necessarily, is in the range of 45 mg to 500 mg. The phytochemically standardized dosage in a thin film piece of a phytochemical can vary depending on the phytochemical and the weight of the film piece. In one embodiment of the invention, the maximum phytochemically standardized dosing is in the range of five to fifty mg of a phytochemical. The number of servings (i.e., the number of thin film pieces) required to achieve a prescribed dosage of a phytochemical typically is one to two film pieces, but can also vary as desired.

While the shape and dimension of thin film pieces can vary as desired, currently preferably the pieces are ¾ of an inch wide by one inch long and are in the shape of a rectangle or of a leaf. The thickness of a film piece presently preferably is in the range of 0.040 to 1.75 micrometers.

The invention reconstitutes a marijuana plant or leaf into a phytochemically standardized form that is orally consumable (for example a thin film of the type discussed herein), a gel or liquid form that can be administered on nasal mucosa, or in a dry form that can be smoked in a pipe or rolled into a cigarette type format to be burned and inhaled into the lungs. A dry form can be produced by removing water or other liquid from the slurry that is produced in accordance with the process described above by grinding and homogenizing a marijuana plant or leaf.

Any desired flavoring or odorant can be incorporated in a reconstituted form of a marijuana plant or leaf. The concentration of such a flavoring or odorant can, for example be in the range of 0.01% to 40.0% by weight, preferably 0.25% to 30.0% by weight, and more preferably 0.5% to 15% by weight.

One advantage of the invention is that it allow governmental authorities to better monitor and enforce the production and sale of medical marijuana.

Another advantage of the invention is that governmental authorities have a quantifiable way to tax marijuana production, and to monitor dosages and usage.

A further advantage of the invention is that it encourages the use of beneficial marijuana phyochemicals without having to smoke either natural marijuana leaves or marijuana in a reconstituted form.

Still another advantage of the invention is that it ensures a more consistent way to deliver potent constituents from marijuana in consistent and repeatable doses.

In another embodiment of the invention, the phytochemically standardized slurry that is produced by cutting, grinding, or pulverizing, and by homogenizing marijuana leaves is not used to produce a thin film, but is instead packaged and sold as is. Prior to such packaging, flavoring, sweeteners, and coloring can, if desired, be added to the slurry, as can any other desired components. The slurry can, for example, be distributed in a pump spray container. Each pump of the spray container dispenses a dose containing a predictable quantity of at least one beneficial phytochemical in marijuana. Or the slurry can be distributed in a bottle without a pump cap, in which cases a particular dose, say one tablespoon, is dispensed and ingested by pouring the slurry from the bottle into a tablespoon and ingesting the slurry in the tablespoon. Regardless of the design of a dispenser used to distribute the slurry, the slurry can be used in cooking, can be dried and smoked, etc.

The following examples are given by way of illustration, and not limitation, of the invention.

### EXAMPLE I

### Production of Thin Film Product

One hundred pounds of fresh picked green live marijuana leaves are harvested and processed in a Fitzpatrick mill to preliminarily cut, grind and pulverize the leaves to produce a coarse slurry that will go through a screen having openings no larger than those found in a 10 mesh screen.

The resulting coarse slurry is directed into an attrition mill to reduce the leaf particulate size to produce a fine slurry that will pass through a screen having openings no larger than those found in a 30 mesh screen.

The resulting fine slurry is further homogenized in a swept surface vessel homogenizer with a homogenizing head for thirty minutes. The homogenizer is turned off. Flavors and sweeteners are added. The homogenizer is turned on for five minutes to produce a homogeneous slurry. The homogeneous slurry is tested to determine the concentration of THC. If necessary, additional THC is added to produce the desired concentration of THC in the slurry.

The resulting phytochemically standardized homogenous slurry is dispensed onto a flat substrate to produce an preliminary film layer that is about 0.030 inch thick. The thickness of the preliminary film layer can be varied as desired. The substrate and film layer are passed through a fifty foot long drying oven at a temperature of 250 to 400 degrees F. for a time period between two to five minutes. The resulting dessicated thin film is cut into strips that are three quarters of an inch wide. The strips are cut into pieces that are one inch long. The pieces are packaged in cassettes, with twenty-eight pieces per cassette.

The amount of tax due a governmental authority(s) is calculated and paid prior to shipment of the cassettes from the production facility.

### EXAMPLE II

### Production of Slurry Product

Two hundred pounds of fresh picked green live marijuana leaves are harvested and processed in a Fitzpatrick mill to preliminarily cut, grind and pulverize the leaves to produce a coarse slurry that will go through a screen having openings no larger than those found in a 10 mesh screen.

The resulting coarse slurry is directed into an attrition mill to reduce the leaf particulate size to produce a fine slurry that will pass through a screen having openings no larger than those found in a 30 mesh screen.

The resulting fine slurry is further homogenized in a swept surface vessel homogenizer with a homogenizing head for thirty minutes. The homogenizer is turned off. Flavors and sweeteners are added. The homogenizer is turned on for five minutes to produce a homogeneous slurry. The homogeneous slurry is tested to determine the concentration of THC. If necessary, additional THC is added to produce the desired concentration of THC in the slurry.

The homogenized phytochemically standardized slurry is pumped into a holding tank. A pumping system is utilized to charge bottles each with a metered quantity of the slurry. The quantity of slurry in each bottle would contain a desired dose(s) of THC. The bottles are labeled and packaged. The tax due on each bottle is calculated and paid to a governmental authority before the bottle is shipped.

### EXAMPLE III

### Production of Tobacco Product

Five hundred pounds of fresh picked green live marijuana leaves are harvested and processed in a Fitzpatrick mill to preliminarily cut, grind and pulverize the leaves to produce a coarse slurry that will go through a screen having openings no larger than those found in a 10 mesh screen.

The resulting coarse slurry is directed into an attrition mill to reduce the leaf particulate size to produce a fine slurry that will pass through a screen having openings no larger than those found in a 30 mesh screen.

The resulting fine slurry is further homogenized in a swept surface vessel homogenizer with a homogenizing head for thirty minutes. The homogenizer is turned off. Flavors and sweeteners are added. The homogenizer is turned on for five minutes to produce a homogeneous of THC. The homogenized slurry is tested to determine the concentration of THC in the slurry. If necessary, additional THC is added to produce the desired concentration of THC in the slurry.

The resulting homogenous phytochemically standardized slurry is dispensed onto a flat substrate to produce an preliminary film layer that is about 0.030 inch thick. The thickness of the preliminary film layer can be varied as desired. The substrate and film layer are passed through a fifty foot long drying oven at a temperature of 250 to 400 degrees F. for a time period between two to five minutes. The resulting dessicated thin film is shredded into a tobacco-like consistency and is packaged in foil-lined pouches to facilitate smoking the phytochemically standardized marijuana thin film.

The amount of tax due a governmental authority(s) is calculated and paid prior to shipment of the cassettes from the production facility.

### EXAMPLE IV

### Culinary Application

The thin film of Example I, the slurry of Example II, or the shredded thin film of Example III are utilized in desired quantities in baking cookies or bread, in roasting chicken, in making lemonade or other drinks, or in the production of other edible products. The thin film, slurry, or shredded thin film can, for purposes of cooking, be sweetened or unsweetened, or flavored or unflavored.

1. A process to produce and dispense a beneficial bioactive phytochemical found in a marijuana plant, comprising the steps of
(a) selecting an area of restricted size in which to grow marijuana;
(b) growing marijuana in said selected area;
(c) harvesting live green leaves from said marijuana plants being grown in said selected area;
(d) determining and recording in a first tangible inventory file the quantity of marijuana plants harvested;
(e) mechanically processing said live green marijuana leaves to produce a standardized homogenized slurry with particulate sufficiently small to pass through a screen having openings no larger than those in a 25 mesh screen, said slurry including natural hemp fibers and the beneficial bioactive phytochemical found in the marijuana plant and uniformly distributed throughout said slurry;
(f) determining the concentration in said slurry of the beneficial bioactive phytochemical;
(g) adding, if necessary, to said slurry an additional quantity of the beneficial bioactive phytochemical to achieve a desired concentration in the slurry of the phytochemical;
(h) dispensing said slurry on a drying surface;
(i) moving said drying surface and said slurry through an oven to remove moisture from said slurry and produce a thin film including natural hemp fibers and the beneficial bioactive phytochemical;
(j) cutting said thin film into film pieces each having a selected dosage of the beneficial bioactive phytochemical;
(k) packaging said film pieces to produce thin film packages each including a selected number of said film pieces;
(l) recording in a second tangible inventory file the number of said thin film packages produced;
(m) paying tax on said thin film packages;
(n) recording in a third tangible inventory file the tax paid on each of said thin film packages sold; and,
(o) auditing said first, second, and third inventory files.

2. A process to produce from a marijuana plant a composition containing a beneficial bioactive phytochemical found in the marijuana plant, comprising the steps of
(a) selecting an area of restricted size in which to grow marijuana;
(b) growing live marijuana plants in said selected area;
(c) harvesting live green leaves from said marijuana plants being grown in said selected area;
(d) determining and recording in a first tangible inventory file the quantity of marijuana plants harvested;
(e) mechanically processing said harvested live green marijuana leaves to produce a standardized homogenized slurry with particulate sufficiently small to pass through a screen having openings no larger than those in a 25 mesh screen, said slurry including natural hemp fibers and the beneficial bioactive phytochemical found in the marijuana plant and uniformly distributed throughout said slurry;
(f) determining the concentration in said slurry of the beneficial bioactive phytochemical; and,
(g) adding, if necessary, to said slurry an additional quantity of the beneficial bioactive phytochemical to achieve a desired concentration in the slurry of the phytochemical.
